(19) United States
(12) Patent Application Publication
(10) Pub. No.: US 2002/0198373 A1
(43) Pub. Date: Dec. 26, 2002

(54) PARTICULATE DRUG CARRIERS

(76) Inventor: IJEOMA FLORENCE UCHEGBU, GLASGOW (GB)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 09/380,854

(22) PCT Filed: Mar. 25, 1998
(86) PCT No.: PCT/GB98/00903

(30) Foreign Application Priority Data
Mar. 25, 1997 (GB) 9706195.6

(51) Int. Cl. C08B 37/08; C07G 1/00; A61K 31/715; A01N 43/04; C08B 3/00; A61K 9/50; A61K 9/127; C08B 11/193

(52) U.S. Cl. 536/58; 514/54; 514/55; 514/59; 424/450; 424/499; 424/279.1; 536/20; 536/84; 536/90; 536/95; 536/112; 536/123.1; 536/123.12

(57) ABSTRACT

The present invention is directed to particulate drug carriers, such as vesicles, formed from polysaccharide derivatives. A polysaccharide bearing at least one non-ionic hydrophilic group attached to an individual monosaccharide unit is hydrophobised to form a derivative bearing at least one long chain alkyl residue. Particle formation is then induced in the presence of cholesterol. The particles are suited for entrapment or conjugation of pharmaceutically active ingredients.

### FIELD OF THE INVENTION

This invention relates to particulate drug carriers formed from polysaccharide derivatives. A polysaccharide bearing at least one non-ionic hydrophilic group attached to the individual monosaccharide units is hydrophobised to form a derivative bearing at least one long chain alkyl residue. Particle formation is then induced in the presence of cholesterol. The particles are suited for entrapment or conjugation of pharmaceutically active ingredients.

### BACKGROUND TO THE INVENTION

Chitosan (N-deacetylated chitin) has been investigated as a drug delivery agent when fabricated into cross-linked microspheres (Thanoo et al, J. Pharma. Pharmacol., 1992, 44, 283-286) and as a coating for liposomes (Henriksen et al, Int. J. Pharm., 1994, 101, 227-236). Chitosan solutions have also been used as penetration enhancers (Aspden et al, Eur. J. Pharm. Sci., 1996, 4, 23-32).

Yoshioka et al (Biosci. Biotech. Biochem., 1993, 57, 1053-1057) have shown that the precipitation (on standing) of a liposome suspension prepared from hydrogenated egg yolk lecithin could be prevented by treating the suspension with an aqueous solution of sulphated N-myristoyl chitosan (S-M-Chitosan). This result is explained by the surface of the liposomes being coated with S-M-chitosan, the ionisation of the sulphate group leading to a negative charge on the liposomes. This in turn causes repulsion between the liposome particles and prevents precipitation. The coating process was not found to destroy the lipid bilayer.

More recently, the same group (in Biosci. Biotech. Biochem., 1995, 59, 1901-1904) have examined the properties of aqueous solutions of chitosan derivatives, namely sulphated N-acyl chitosan (S-Cₙ-chitosan) having varying lengths of alkyl chain. S-Cₙ-chitosans from C₂ to C₁₄ dissolved completely in water to form transparent solutions. S-C₁₆-chitosan was also prepared but its properties were not examined since the aqueous mixture formed was not transparent and it was believed that an aggregate such as a liquid crystal had been formed. The solubilising capacity of the aqueous S-Cₙ-chitosan solutions towards a hydrophobic substance (azobenzene) was examined and it was found that solubility increased sharply with increasing carbon number above C₁₀. It was concluded by the authors that the long alkyl chains were aggregated to form micelles able to dissolve the azobenzene molecules. The micelles formed are described as “polymer micelles”, although the type of micelle formed was not ascertained. The “polymer micelles” are suggested for use as drug carriers.

Sunamoto et al (Chem. Lett., 1991, 1263-1266) have reported that palmitoyl- or cholesterol-substituted derivatives of polysaccharides such as pullulan, amylopectin and dextran form self-aggregates in aqueous solution. A palmitoyl pullulan derivative substituted to 5.4 palmitoyl groups per 100 glucose units is reported, together with cholesterol-substituted pullulans having varying degrees of substitution. Investigation of the interaction between the pullulan derivatives and a fluorescent probe showed that the pullulan derivatives formed polymer self-aggregates above a critical concentration. The driving force for the aggregation is ascribed to a hydrophobic interaction between hydrophobic moieties and it is noted that the palmitoyl group was less effective for forming the self-aggregates. The aggregates are described as having the capacity to encapsulate various substances by hydrophobic interaction, for example drugs, proteins and nucleic acids.

The same group also reports (in Macromolecules, 1993, 26, 3062-3068) on the synthesis and solution properties of a non-ionic cholesterol-modified pullulan derivative (CHP) in water. In this work, pullulan was substituted by 1.6 cholesterol groups per 100 anhydroglucoside units. The authors state that the CHP self-aggregates formed relatively monodispersive particles and it is suggested that one CHP self-aggregate consists of approximately 13 CHP molecules. Experimental data suggests that the hydrophobic core of the CHP aggregates is completely and stably covered by the hydrophilic shell of the polysaccharide skeleton, forming colloidally stable nanoparticles above the critical concentration. The binding of various fluorescent probes was investigated and shown to increase with an increase in hydrophobicity of the probe. It is therefore concluded that the main driving force for complexation is a hydrophobic interaction.

These researches have further described (in Chem. Lett., 1995, 707-708) the complexation of the hydrogel nanoparticle formed by self-assembly of CHP with 5-10 insulin monomers in water. The number of complexed insulin molecules increased with an increase in the substitution degree of the cholesterol group of CHP. The insulin is stated as being complexed deeply inside the amphiphilic hydrogel matrix of the nanoparticle in which the hydrophobic microdomain of the associating cholesterol forms non-covalent crosslinks of gel structure. The number of crosslinks of one nanoparticle increases with an increase in the number of substitution degree of cholesterol, leading to an increase in the binding site for insulin.

Finally, the same group reports (in J. Am. Chem. Soc., 1996, 118, 6110-6115) a study of the complexation between CHP self-aggregate and bovine serum albumin (BSA). In all cases, approximately one BSA molecule was complexed by one nanoparticle of CHP self-aggregate, irrespective of the structure of the CHP self-aggregate. Unfolding of BSA by thermal means or by a denaturant such as urea was largely suppressed on complexation. This stabilisation of BSA upon complexation is ascribed to the formation of multiple non-covalent interactions between BSA and the hydrogel of CHP self-aggregate.

### SUMMARY OF THE INVENTION

According to the present invention, there is provided a compound which is a polysaccharide derivative bearing at least one non-ionic hydrophilic group and at least one hydrophobic group per molecule wherein said hydrophobic group is attached to the individual monosaccharide units and said hydrophobic group contains a C₁₂₋₂₄ alkyl, alkenyl, alkynyl or acyl residue.

The non-ionic hydrophilic group is preferably a group of the formula R¹, wherein R¹ is selected from mono- and oligo-hydroxy C₁₋₆ alkyl, mono- and oligo-hydroxy substituted C₂₋₆ acyl, C₁₋₂ alkoxy alkyl optionally having one or more hydroxy groups substituted on the alkoxy or alkylene groups, oligo- or poly-(oxa C₁₋₃ alkylene) preferably polyoxyethylene comprising up to about 120 ethylene oxide units (i.e. up to a molecular weight of 5000), and C₁₋₄ alkyl (oligo- or poly-oxa C₁₋₃ alkylene) optionally hydroxy substituted preferably oligo- or polyglycerol ethers such as those described in GB-A-1,529,625, for example containing up to 10 glycerol units; and wherein R¹ is joined via an ether linkage to a saccharide unit of the polysaccharide. It is to be understood herein that the term acyl includes alkenoyl and alkynoyl groups as well as alkanoyl groups.

The requirement that the hydrophilic group is non-ionic is an important feature since a charged ionic group such as sulphate would repel anionic DNA which, in one embodiment, is associated with the particles as a means for gene delivery or vaccination.

The polysaccharide derivative is preferably a derivative of chitosan, pullulan or dextran and most preferably comprises 1,4-linked saccharide units. Normally, substitution by the non-ionic hydrophilic moiety occurs at the C6 position of a saccharide unit.

The hydrophobic group is preferably joined to a saccharide unit by an amide, ester, ether or amine linkage, most preferably by an amide linkage. In a further preferred embodiment, this group is substituted at the C2 position in a 1,4-linked saccharide unit.

The compound has a degree of substitution by non-ionic hydrophilic groups in the range 0.1-1.5, preferably greater than 0.9 and most preferably 1 per saccharide unit.

The ratio of hydrophilic:hydrophobic groups in the compounds of this invention is in the range 100:1 to 1:2, preferably between 10:1 and 2:1 more preferably 5:1 and 2:1. Compounds having a degree of hydrophobic substitution of 0.5 or above per hydrophilic group are found to be difficult to disperse due to the high hydrophobic burden. Consequently, compounds having a degree of substitution of 0.25 or less are preferred.

A preferred range of compounds according to the present invention are the N-substituted derivatives of poly-amino glycans most preferably N-acyl glycol chitosans, especially N-palmitoyl glycol chitosan (poly[β(1-4)-2-deoxy-2-hexadecanamido-6-0-(2-hydroxyethyl)-D-glucopyranose]. In this case, the presence of free amino groups is advantageous from a point of view of permitting complexing with anionic DNA. In addition, such groups could be used for the conjugation of drug molecules.

In a preferred embodiment, the compound has the formula:

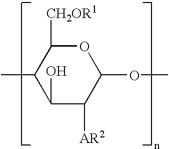

wherein each R¹ is selected from hydrogen, mono- and oligo-hydroxy C₁₋₆ alkyl, mono- and oligo-hydroxy substituted C₂₋₆ acyl, C₁₋₂ alkoxy alkyl optionally having one or more hydroxy groups substituted on the alkoxy or alkylene groups, oligo- or poly-(oxa C₁₋₃ alkylene) such as polyoxyethylene comprising up to about 120 ethylene oxide units and C₁₋₄ alkyl (oligo- or poly-oxa C₁₋₃ alkylene) optionally hydroxy substituted such as polyglycerol ethers, for example containing up to 10 glycerol units, provided that at least one of the groups R¹ is other than hydrogen;

A is —NH—, or —O—;

each R² is selected from hydrogen, C₁₂₋₂₄ alkyl, alkanoyl, -alkenyl, alkenoyl, -alkynyl or alkynoyl, provided that at least one of the groups R² is other than hydrogen; and

n is 5-2000.

Preferably, the group R¹ has the formula —CH₂CH₂OH or —CH₂CH(OH)CH₂OH, R² is C₁₆₋₁₈ acyl and A is —NH—.

The compounds may be formed according to any of the standard techniques described in the prior art for the derivatisation of polysaccharides (see for example, the references by Yoshioka et al -op cit). The technique may involve derivatisation of a polysaccharide starting material by a hydrophilic group in a first step, followed by a second step comprising attachment of a hydrophobic group or vice-versa. Alternatively, commercially-available polysaccharide derivatives already possessing a hydrophilic group may be hydrophobised using standard techniques to form a compound according to this invention.

The compounds described are used in combination with cholesterol or a derivative thereof to form particles. In the absence of cholesterol, particle formation does not occur and the material precipitates. Consequently, the presence of cholesterol is required to promote self-assembly of the polysaccharide derivatives to form particles.

The particles are made by techniques similar to those used to form liposomes and niosomes, for instance by blending the compounds in an organic solvent and then contacting the dried mixture with an aqueous solution, optionally followed by a particle size reduction step.

The particles formed may be suspended in an aqueous vehicle or alternatively may be isolated in a dry state. The particles may optionally incorporate a steric stabilizer, for instance a non-ionic amphiphilic compound, preferably a poly-24-oxyethylene cholesteryl ether. The particles may be micro or nano-particulate, nano-particles being formed preferably in the presence of the steric stabilizer. In this case, the steric stabilizer is incorporated into the structure of the particle.

The particles preferably also comprise an associated pharmaceutically active ingredient. The active ingredient may be water soluble, in which case it will be associated with the hydrophilic regions of the particle, or water insoluble and consequently associated with the hydrophobic regions of the particle.

Such an ingredient is preferably physically entrapped within the particle but may also be held by covalent conjugation. The pharmaceutically active ingredient may be a peptide or protein therapeutic compound. A further preferred alternative for the pharmaceutically active compound is nucleic acid (eg. DNA), preferably in the form of a gene for gene therapy or gene vaccination.

These drug carriers may be used for the treatment of a human or animal by therapy, in particular for oral drug delivery of peptides or proteins or as gene delivery vectors. It is envisaged that this drug delivery system will also be useful when used via the intravenous, intramuscular, intraperitional or topical (inhalation, intranasal, application to the skin) routes.

The novel compounds according to this invention may also be used for the coating of pre-formed liposomes or niosomes for instance which are drug carriers suspended in an aqueous carrier.

The invention will now be further illustrated with reference to the following non-limiting examples, wherein the fluorescent aqueous marker 5(6)-carboxy fluoroscein (CF) is used as a model drug and with reference to the figures in which:

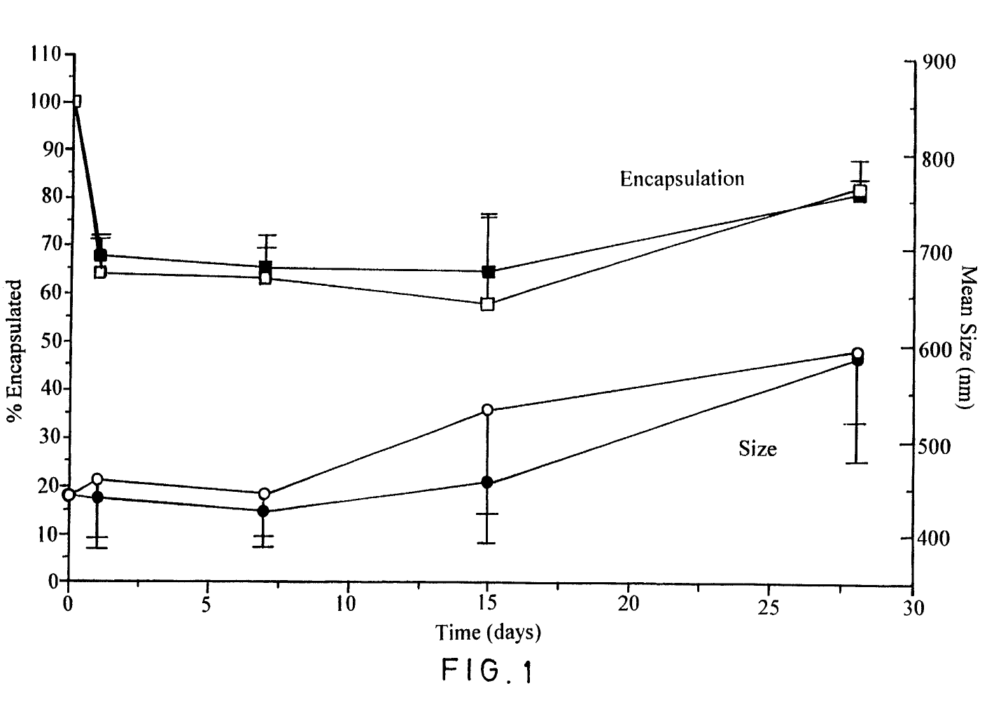

FIG. 1 shows the stability of bleomycin GCP41 based vesicles after storage at 4° C. (●, ▪) and room temperature 16-25° C. (◯, □). ▪, □=% encapsulation, ●, ◯=mean size. Data points=mean±s.d., n=3;

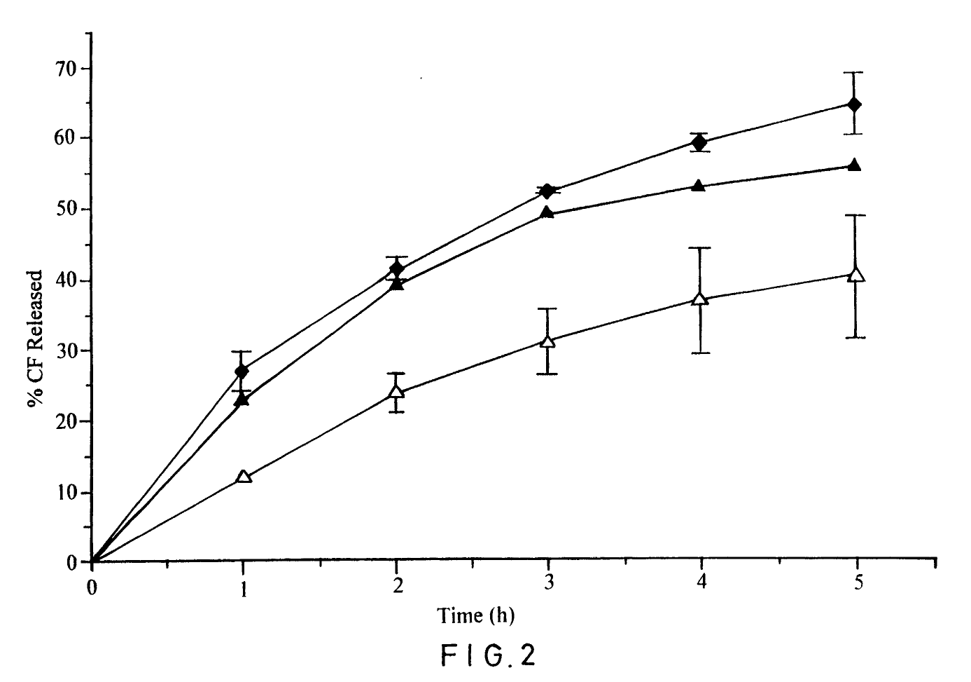

FIG. 2 shows the release of 5(6)-carboxyfluorescein from GCP41, cholesterol vesicles. Data points=mean of 3 determinations. Δ=palmitoyl glycol chitosan based vesicles (mean±s.d., n=6), ▴=Span 60 vesicles (mean, n=3), ♦=5(6)-carboxyfluorescein solution;

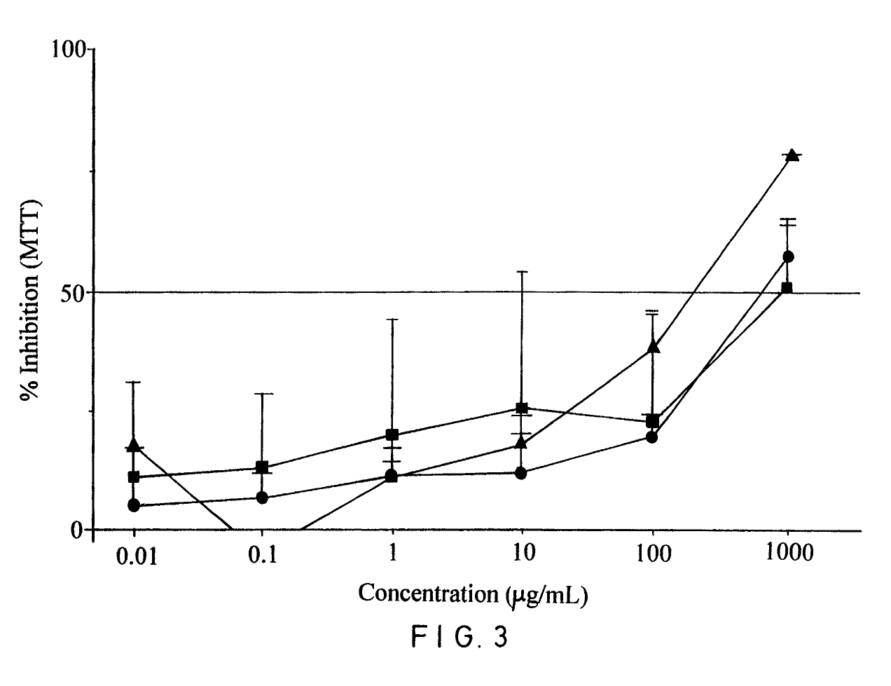

FIG. 3 shows the biocompatibility of GCP41 based vesicles against 3 cell lines, ▪=A549, ●=A431, ▴=A2780. Data points=mean±s.d., n=3; and

### DETAILED DESCRIPTION OF THE INVENTION

### EXAMPLE 1

### Synthesis of N-Palmitoyl Glycol Chitosan (Poly[β(1-4)-2-Deoxy-2-Hexadecanamide-6-0-(2-Hydroxyethyl)-D-Glucopyranose]

a) GCP41 (4:1 Initial Ratio of Glycol Chitosan: Palmitoyl Units)

200 mg glycol chitosan (GC) and 150 mg sodium bicarbonate was dissolved in 35 mL water. 10 mL absolute ethanol was added, followed by a drop-wise addition of a solution of 79 mg palmitoyl N-hydroxysuccinimide ester dissolved in 60 mL absolute ethanol. Addition of the palymitoyl N-hydroxysuccinimide ester was carried out with stirring over 30 min. The reaction mixture was initially cloudy but turned clear after about 1 h. The reaction mixture was left to stir for 72 h. After this time, 100 mL acetone was added with formation of a slight precipitate. This mixture was then evaporated to reduced volume under reduced pressure at 60° C. The resuting liquid was extracted with 3 volumes of ether and exhaustively dialysed against water for 24 h. The dialyzed mixture was freeze dried to give a white fluffy cotton wool like substance.

b) GCP21 (Initial 2:1 Ratio of Glycol Chitosan: Palmitoyl Units)

200 mg glycol chitosan (GC) and 150 mg sodium bicarbonate was dissolved in 35 mL water. 10 mL absolute ethanol was added, followed by a drop-wise addition of a solution of 150 mg palmitoyl N-hydroxysuccinimide ester dissolved in 120 mL absolute ethanol. Addition of the palmitoyl N-hydroxysuccinimide ester was carried out with stirring over 30 min. The reaction mixture was initially cloudy but turned clear after about 6 h. The reaction mixture was left to stir for 72 h. After this time, 100 mL acetone was added with formation of a slight precipitate. This mixture was then evaporated to reduced volume under reduced pressure at 60° C. The resulting liquid was extracted with 3 volumes of diethyl ether and exhaustively dialyzed against water for 24 h. The dialyzed mixture was freeze dried to a white fluffy cotton wool like substance. This was washed with water and the sticky mass freeze dried to give a fluffy cotton wool like substance.

c) Characterisation of GCP41

¹H NMR

Glycol chitosan is moderately soluble in water (2 mg mL⁻¹) and ¹H NMR (with integration) and H¹-H¹COSY experiments were carried out on glycol chitosan in (D₂O, Sigma Chemical Co., UK) and GCP41 in a CD₃OD/D₂O mixture using a Bruker AMZ 400 MHz in order to assign the non-exchangeable coupled protons.

FT-IT

FT-IR was performed in potassium bromide discs on a Mattson Galaxy FT-IR.

The level of hydrophobic modification in GCP41 and the original level of acetylation in glycol chitosan were assessed by ¹H NMR (Vårum et al 1991, Yoshioka et al 1993). In this way the batch of glycol chitosan (Sigma Chemical Co, UK—105H0111) that was used was found to be one third acetylated. Proton assignments, δ0.86 p.p.m=CH₃ (palmitoyl) δ1.25 p.p.m=CH₂ (palmitoyl), δ1.89 p.p.m=CH₂ (palmitoyl—shielded by carbonyl), δ2.13 p.p.m=CH₃ (acetyl-GCP41), δ2.14 p.p.m.=CH₂ (adjacent to carbonyl protons), δ1.99 p.p.m=CH₃ (acetyl-glycol chitosan), δ2.71 p.p.m=CH (C2 sugar proton-GCP41), δ2.64 p.p.m=CH (C2 sugar proton-GCP41), δ3.31 p.p.m=methanol protons, δ3.3-4.0 p.p.m=non-exchangeable sugar protons, δ4.4 p.p.m=water protons. The level of hydrophobic modification in GCP41 was assessed by using the ratio of non-exchangeable C2 protons to methyl protons (spectrum b) and was found to be 14.48±2.88% (mean±s.d., n=3) with values lying between 11 and 16 mole %. The ratio of N-acetyl protons, C2 sugar protons, 9 additional sugar/glycol non-exchangeable protons remains at (˜1:1:10) in all three spectra.

GCP41 was insoluble yet dispersible in D₂O to give a cloudy liquid which remained without a sediment for at least 4 weeks. The ¹H NMR spectra of a fresh sample of this dispersion is devoid of signals for the fatty acid side chain protons. This suggests that palmitoyl glycol chitosan in water adopts an orientation in which the fatty acid side chains exist in hydrophobic domains separated from the hydrophilic part of the polymer. The acetyl group appears to be an integral part of the hydrophilic portion of the molecule in the modified polymer as signals for the acetyl groups are clearly seen in the GCP41-D₂O spectra. Hence there was no co-operative association between the acetyl group and the hydrophobic side chains when palmitoyl glycol chitosan was dispersed in water. Freeze fracture electron microscopy did not reveal the existence of any discernible particulate matter in this cloudy liquid.

The GCP41 FT-IR spectrum revealed a sharpening of the amide peak at 1648 cm⁻¹. The starting material glycol chitosan contains a relatively smaller amide peak at 1653 cm⁻¹.

### EXAMPLE 2

### Preparation and Characterization of GCP41 and GCP21 Micro- and Nano-Particles

a) GCP21—Cholesterol Particles

7.2 mg cholesterol was dissolved in 10 mL chloroform. To this solution was added 12.2 mg GCP21. The organic solvent was removed under vacuum and the solid deposit dried under a stream of nitrogen. 2 mL of aqueous CF (5 mM) was added to this solid deposit and the mixture shaken for 1 h at 70° C. to form a homogenous dispersion of micro-particles.

0.1 mL of this dispersion was then fractionated over a Sephadex G50 column (205×8 mm) and the sample eluting in the void volume collected. This sample was sized in a Malvern Mastersizer. Assay for entrapped material was carried out by solubilizing the particles in isopropanol (0.1 mL dispersion to 1 mL isopropanol). CF was then assayed by fluorometry (exc.=486 nm, em.=514 nm).

b) GCP21—Cholesterol—Solulan C24 Particles

6.2 mg cholesterol and 5.4 mg Solulan C24 were dissolved in 10 mL chloroform. To this solution was added 11 mg GCP21. The organic solvent was removed under vacuum and the solid deposit dried under a stream of nitrogen. 2 mL of aqueous CF (5 mM) was added to this solid deposit and the mixture shaken for 1 h at 70° C. to form a homogenous dispersion of micro-particles.

Nano-particles were prepared by filtration of this dispersion (0.22 μm).

0.1 mL of these dispersions was then fractionated over a Sephadex G50 column (205×8 mm) and the sample eluting in the void volume collected. This sample was sized in a Malvern Mastersizer or Autosizer depending on the particle size. Assay for entrapped material was carried out by solubilizing the particles in isopropanol (0.1 mL dispersion to 1 mL isopropanol). CF was then assayed by fluorometry (exc.=486,em.+514 nm).

c) GCP41—Cholesterol Particles

7.3 mg cholesterol was dissolved in 10 mL chloroform. To this solution was added 19.8 mg GCP41. The organic solvent was removed under vacuum and the sold deposit dried under a stream of nitrogen. 2 mL of aqueous CF (5 mM) was added to this solid deposit and the mixture shaken for 1 h at 70° C. to form a homogenous dispersion of micro-particles.

0.1 mL of this dispersion was then fractionated over a Sephadex G50 column (205×8 mm) and the sample eluting in the void volume collected. This sample was sized in a Malvern Mastersizer. Assay for entrapped material was carried out by solubilizing the particles in isopropanol (0.1 mL dispersion to 1 mL isopropanol). CF was then assayed by fluorometry (exc.=486 nm, em.=514 nm).

d) GCP41—Cholesterol—Solulan C24 Particles

6.5 mg cholesterol and 5.4 mg Solulan C24 were dissolved in 10 mL chloroform. To this solution was added 17.3 mg GCP41. The organic solvent was removed under vacuum and the solid deposit dried under a stream of nitrogen. 2 mL of aqueous CF (5 mM) was added to this solid deposit and the mixture shaken for 1 h at 70° C. to form a homogenous dispersion of micro-particles.

Nano-particles were formed by filtration of this dispersion (0.22 μm).

0.1 mL of this dispersion was then fractionated over a Sephadex G50 column (205×8 mm) and the sample eluting in the void volume collected. This sample was sized in a Malvern Mastersizer or Autosizer. Assay for entrapped material was carried out by solubilizing the particles in isopropanol (0.1 mL dispersion to 1 mL isopropanol). CF was then assayed by fluorometry (exc.=486 nm, em.=514 nm).

The sizes and encapsulation efficiencies are given in Table 1.

### EXAMPLE 3

### Preparation of Bleomycin Entrapped Vesicles

GCP41 vesicles were prepared by the sonication of GCP41 (8 mg) and cholesterol (4 mg, Sigma Chemical Co., UK) in water for 2×2 minutes with the instrument set at 20% of its maximum capacity. Bleomycin GCP41 vesicles were prepared by sonicating GCP41 (8 mg) and cholesterol (4 mg) in 2 mL ammonium sulphate (0.12M, Sigma Chemical Co., UK). Unentrapped ammonium sulphate was removed by ultracentrifugation (150,000 g×1 h—MSE 75 superspeed). Vesicles were then incubated for 1 h at 60° C. with bleomycin (Lundbeck, UK) solution (2 mL 6U mL⁻¹) and left to stand overnight at room temperature. Unentrapped bleomycin was also removed by ultracentrifugation (150,000 g×1 h) and entrapment was measured by disrupting the vesicles in 10×volume isopropanol (Rathburn Chemical Co., UK) followed by ultraviolet absorption spectrophotometry at 254 nm (Unicam UV-1).

On storage at room temperature there was an initial loss of bleomycin although over 60% of the drug is retained within the vesicles (see FIG. 1). Particle size is also seen to change very little. The stability data suggests that there is a loosely bound and a tightly bound fraction of bleomycin associating with GCP41 vesicles. The tightly bound fraction is presumed to be that fraction of bleomycin that traverses the membrane of the polymeric vesicle and actually accumulates within it in response to the ammonium sulphate gradient.

### EXAMPLE 4

### 5(6)-Carboxyfluorescein Release from Vesicles

Vesicles were prepared as described in Example 3 from GCP41 (16 mg) and cholesterol (8 mg) except that the hydrating solution was 4 mL 5(6)-carboxyfluorescein (5.03 mM, Sigma Chemical Co., UK). Sorbitan monostearate vesicles were prepared by hydrating sorbitan monostearate (24 mg, Sigma Chemical Co., UK), cholesterol and poly-24-oxyethylene cholesteryl ether (16 mg, D. F. Anstead, UK) in the presence of 4 mL 5(6)-carboxyfluorescein (5.03 mM). Unentrapped material was again removed by ultracentrifugation as described in Example 3. The release of 5(6)-carboxyfluorescein from GCP41 and sorbitan monostearate vesicles was monitored as follows. A 1:2 mixture of the vesicles and 2% w/w bile salts (sodium cholate and sodium deoxycholate, Sigma Chemical Co., UK) was placed in a 5 cm piece of Visking tubing (Mw cut off 12,000-14,000) sealed at both ends. This mixture was dialysed against a 13-fold volume of the bile salts solution. 5(6)-carboxyfluorescein external to the dialysis tubing was monitored fluorimetrically (exc. 486, em.=514 nm, Perkin Elmer LS-5) at regular time intervals. A 1:2 mixture of 5(6)-carboxyfluorescein in phosphate buffered saline (PBS, pH=7.4) (0.5 mL) and 2% w/w bile salts (1 mL) was included as a control.

Using the release of the small molecular weight compound 5(6)-carboxyfluorescein (Mw=387) as a marker for vesicle integrity, these polymeric vesicles are found to be more resistant to attack by detergents than vesicles prepared from the non-ionic surfactant sorbitan monostearate (see FIG. 2). This is believed to be due to the difficulty the soluble bile salt surfactants have in inserting into a polymeric bilayer as opposed to the ease of insertion into a bilayer resulting from the self-assembly of monomers.

### EXAMPLE5

### Biocompatibility and Haemocompatibility Studies

Biocompatibility Studies

Cytotoxicity was evaluated by the IC50 value in a standard MTT based assay (Freshney et al Clulture of Animal Cells, 3rd edition, Wiley-Liss, New York, 1994). Depending on the growth rate, 0.5-2.0×10³ cells per well were seeded into 96 well plates and incubated for 24 h. Serial dilutions of the suspensions were added and incubated with the cells for 12 h. The suspensions were replaced with fresh medium and the cells were incubated with repeated feeding for 72 h. 50 mg mL⁻¹ MTT [3-(4,5-dimethylthiazol-2-yl)-2,5-diphenyltetrazolium bromide, 50 μL, Sigma Chemical Co., UK) was added to each well. After incubation for 4 h in the dark, the medium and MTT solution was removed and the cells were lysed in DMSO (200 μL, Sigma Chemical Co., UK). Following the addition Sorensen's glycine buffer (25 μL) the absorption was measured at 570 nm.

Haemocompatibility Studies

Freshly drawn human blood was centrifuged (3,000 g) to separate the red blood cells. These were washed in PBS (pH=7.4) and weighed. 3 g of the erythrocyte pellet was dispersed in 100 mL PBS (pH=7.4) and incubated for 5 h with various concentrations of GCP41, cholesterol vesicles prepared as described above or DOTAP vesicles (Sigma Chemical Co., UK). Haemolysis was assessed by centrifugation (3,000 g) to isolate the released haemoglobin, addition of 2×volume of isopropanol to the supernatant and the measurement of the absorbance (570 nm).

GCP41 vesicles were biocompatible with 3 human cell lines A2780 (ovarian cancer cell line), A549 (lung carcinoma) and A431 (epidermoid carcinoma) with no toxicity evident at concentrations of GCP41 below 150 μg mL⁻¹ and IC50 values of 0.2, 1.0 and 1.0 mg mL⁻¹ respectively (FIG. 3). GPC41 vesicles showed good haemocompatibility with human erythrocytes and an ability to modulate the haemolytic activity of N-[1-(2,3-dioleoyloxy)propyl]-N,N,N-trimethylammonium methylsulphate (DOTAP)—the DNA transfection agent (Porteous et al, (1997) Gene Therapy 4, 210-218) (Table 2). These biocompatibility data are in good agreement with that reported for soluble glycol chitosan against the B16F10 cell line and rat erythrocytes (Carreno-Gomez and Duncan (1997) Int. J. Pharm. 148, 231-240.

### EXAMPLE 6

### Preparation of Insulin and LHRH Entrapped Vesicles

GCP21 was prepared according to Example 1

a) Insulin. GCP21 vesicles were prepared by sonication of a mixture of GCP21 (8 mg) and cholesterol (4 mg) in 2 mL water. GCP21 vesicles were loaded with insulin by either incubating 1 ml of the vesicle dispersion with 1 ml of insulin (160 IU mL⁻¹) for 16 h at room temperature or by the use of the dehydration-rehydration (DRV) method (Kirby C., Gregoriadis G (1984) Biotechnology 979-984) in which insulin vesicles mixtures, as described above, were lyophilised overnight and subsequently rehydrated to 2 mL volume. The amount of insulin encapsulated was assessed by HPLC after separation of encapsulated insulin from the unencapsulated material by ultracentrifugation (150,000 g) and disruption of the vesicles with isopropanol (1 ml isopropanol to 1 ml of the vesicle dispersion). Vesicles were also sized by photon correlation spectroscopy and the zeta potential of the dispersion measured.

b) Lutenizing hormone releasing hormone (LHRH) LHRH was loaded onto GCP21 vesicles by the use of ammonium sulphate gradients (Haran, G et al (1993) Biochym. Biophys. Acta 1151, 201-205). Vesicles encapsulating ammonium sulphate were prepared by sonicating GP21 (8 mg), cholesterol (4 mg) mixtures in 2 mL of a solution of ammonium sulphate (0.03M). Unentrapped ammonium sulphate was separated by ultracentrifugation (150,000 g) and the pelleted ammonium sulphate vesicles were incubated with 2 mL LHRH (2.5 mg mL⁻¹). Unentrapped LHRH was also removed by ultracentrifugation (150,000 g for one hour). These vesicles were also sized by photon correlation spectroscopy.

a) Insulin. Insulin GCP21 vesicles could be prepared by incubating pre-formed GCP21 vesicles with insulin at room temperature for approximately 16 h (Table 3). No improvement in the level of insulin associated with the vesicles was observed with the DRV method. The zeta potential of the GCP21 vesicles increased from −5 mV to +10 mV on loading with insulin, indicating that the insulin associates with the surface of the vesicles to a certain extent.

b) LHRH. LHRH vesicles could also be prepared by the use of ammonium sulphate gradients (Table 4).

TABLE 1
Size and CF encapsulation efficient of GCP21 and
GCP41 particles.

| | | % CF |
|---|---|---|
| PARTICLE | SIZE | ENCAPSULATION |
| GCP21/Cholesterol micro- | 34.6 μm | 7.4% |
| particles | | |
| GCP21/Cholesterol/Solulan | 30.7 μm | 4.6% |
| C24 micro-particles | | |
| GCP41/Cholesterol micro- | nd | 9.3% |
| particles | | |
| GCP41/Cholesterol/Solulan | nd | 4.2% |
| C24 micro-particles | | |
| GCP21/Cholesterol/Solulan | 325 nm | 6.88% |
| C24 nano-particles | | |
| GCP41/Cholesterol/Solulan | 333 nm | 4.6% |
| C24 nano-particles | | |

TABLE 2
The haemocompatibility of GCP41 vesicles

| | | % Haemolysis |
|---|---|---|
| Formulation | Erythrocyte, polymer/DOTAP ratio | (n = 3)* |
| DOTAP | 30 (erythrocyte, DOTAP ratio) | 101.4 ± 20.4 |
| DOTAP | 300 (erythrocyte, DOTAP ratio) | 71.0 ± 10.6 |
| GCP41, cholesterol | 30 (eythrocyte, GCP41 ratio) | 4.2 ± 1.6 |
| (8:4) | | |
| GCP41, cholesterol, | 60 (erythrocyte, DOTAP ratio) | 10.7 ± 1.3 |
| DOTAP | | |
| (6:2:1) | | |
| GCP41, cholesterol, | 600 (erythrocyte, DOTAP ratio) | 6.6 ± 1.8 |
| DOTAP | | |
| (6:2:1) | | |

*Haemocompatibility is expressed with respect to 100% haemolysis produced by an erythrocyte, triton X-100 weight ratio of 3:1 and 0% haemolysis produced by PBS (pH = 7.4).

TABLE 3
The size and encapsulation efficiency of insulin GCP21 vesicles

| Method of | % Encapsulation | Mean Insulin, | Size (nm, | Zeta Potential |
|---|---|---|---|---|
| Preparation | (mean ± s.d.) | GP21 ratio (IU mg⁻¹) | (mean ± s.d.) | (mV, mean ± s.d.) |
| Empty GCP21 | — | — | 559 ± 78 | — |
| vesicles | | | | |
| Incubation at | 16.21 ± 0.70 | 3.24 | — | — |
| room | | | | |
| temperature | | | | |
| DRV method | 15.12 ± 0.62 | 3.02 | 945 ± 20 | 10.13 ± 4.82 |

TABLE 4
The encapsulation of LHRH in GCP21 vesicles

| | LHRH, GCP21 ratio |
|---|---|
| % Encapsulation | (mg mg⁻¹) |
| 57.5 | 0.54 |

1. A composition comprising particles formed from a compound which is a polysaccharide derivative bearing at least one non-ionic hydrophilic group and at least one hydrophobic group per molecule wherein said hydrophilic group is attached to the individual monosaccharide units and said hydrophobic group contains a C₁₂₋₂₄ alkyl, alkenyl, alkynyl or acyl residue.

2. A composition according to claim 1 wherein the non-ionic hydrophilic group is a group R¹, where R¹ is selected from mono- and oligo-hydroxy C₁₋₆ alkyl, mono- and oligo-hydroxy substituted C₂₋₆ acyl, C₁₋₂ alkoxy alkyl optionally having one or more hydroxy groups substituted on the alkoxy or alkylene groups, oligo- or poly-(oxa C₁₋₃ alkylene) such as polyoxyethylene comprising up to about 120 ethylene oxide units and C₁₋₄ alkyl (oligo- or poly-oxa C₁₋₃ alkylene) optionally hydroxy substituted such as oligo- or polyglycerol ethers; and wherein R¹ is joined via and ether linkage to a saccharide unit of the polysaccharide.

3. A composition according to claim 1 or claim 2 wherein the polysaccharide has 1,4-linked saccharide units.

4. A composition according to claim 3 wherein each non-ionic hydrophilic group is substituted at the C6 position of a saccharide unit.

5. A composition according to claim 3 or claim 4 in which the hydrophobic group is substituted at the C2 position.

6. A composition according to any preceding claim wherein the degree of substitution by non-ionic hydrophilic groups is 0.1-1.5, preferably at least 0.9 per saccharide unit.

7. A composition according to any preceding claim wherein the ratio of hydrophilic: hydrophobic groups is in the range 100:1 to 1:2, preferably between 10:1 and 2:1, more preferably between 5:1 and 2:1.

8. A composition according to any preceding claim wherein the hydrophobic group is joined to a saccharide unit by an amide, ester, ether or amine linkage.

9. A composition according to any preceding claim wherein the polysaccharide is a derivative of chitosan, pullulan or dextran.

10. A composition according to any preceding claim which is an N-substituted derivative of a poly-amino glycan.

11. A composition according to claim 10 which is an N-acyl glycol chitosan, preferably N-palmitoyl glycol chitosan.

12. A composition comprising particles formed from a compound having the formula: 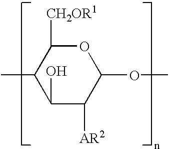
wherein each R¹ is selected from hydrogen, mono- and oligo-hydroxy C₁₋₆ alkyl, mono- and oligo-hydroxy substituted C₂₋₆ acyl, C₁₋₂ alkoxy alkyl optionally having one or more hydroxy groups substituted on the alkoxy or alkylene groups, oligo- or poly-(oxa C₁₋₃ alkylene) such as polyoxyethylene comprising up to about 120 ethylene oxide units and C₁₋₄ alkyl (oligo- or poly-oxa C₁₋₃ alkylene) optionally hydroxy substituted such as polyglycerol ethers, for example containing up to 10 glycerol units, provided that at least one of the groups R¹ is other than hydrogen;
A is —NH— or —O—;
R² is selected from hydrogen, C₁₂₋₂₄ alkyl, -alkanoyl, -alkenyl -alkenoyl, -alkynyl or alkynoyl, provided that at least one of the groups R² is other than hydrogen; and
n is 5-2000.

13. A composition according to claim 12 wherein R¹ is —CH₂CH₂OH or —CH₂CH(OH)CH₂OH.

14. A composition according to claim 12 or claim 13 wherein R² is C₁₆₋₁₈ acyl.

15. A composition according to any of claims 12 to 14 wherein A is —NH—.

16. A composition according to any preceding claim which comprises an aqueous vehicle in which the particles are suspended.

17. A composition according to any preceding claim which additionally comprises cholesterol or a derivative thereof.

18. A composition according to claim 17 which further comprises a steric stabilizer, preferably a non-ionic amphiphilic compound, most preferably a poly-24-oxyethylene cholesteryl ether.

19. A pharmaceutical composition comprising a composition according to any preceding claim and a pharmacologically acceptable carrier.

20. A composition according to any preceding claim which comprises a pharmaceutically active ingredient associated with the particles.

21. A composition according to claim 20 which comprises an entrapped pharmaceutically active ingredient.

22. A composition according to either of claims 20 and 21 which comprises a covalently conjugated pharmaceutically active ingredient.

23. A composition according to any of claims 20 to 22 in which the pharmaceutically active ingredient is a peptide or protein therapeutic compound or DNA, preferably a gene for gene therapy or gene vaccination.

24. A composition or compound according to any of claims 1 to 18 for use in a pharmaceutical composition.

25. Use of a composition or compound according to any of claims 1 to 18 in the manufacture of a medicament for use in therapy.

26. Use of a composition or compound according to any of claims 1 to 18 and a pharmaceutically active ingredient in a method of manufacturing a medicament for use in therapy.
